(19) United States
(12) Patent Application Publication
(10) Pub. No.: US 2008/0207668 A1
(43) Pub. Date: Aug. 28, 2008

(54) PHARMACEUTICAL COMPOSITIONS OF HYDROMORPHONE FOR PREVENTION OF OVERDOSE OR ABUSE

(75) Inventor: James Scott Moncrief, Christiansburg, VA (US)

Correspondence Address: DARBY & DARBY P.C., P.O. BOX 770, Church Street Station, New York, NY, 10008-0770, US

(73) Assignee: New River Pharmaceuticals Inc., Radford, VA (US)

(21) Appl. No.: 11/868,283

(22) Filed: Oct. 5, 2007

Related U.S. Application Data
(60) Provisional application No. 60/849,774, filed on Oct. 6, 2006.

(51) Int. Cl. A61K 47/48; A61K 31/485; C07K 5/00; A61P 25/00; C07D 489/00

(52) U.S. Cl. 514/282; 530/300; 546/45

(57) ABSTRACT

The invention relates to compounds, compositions and methods comprised of a chemical moiety attached to hydromorphone. The invention provides embodiments that provide a decrease in the potential of hydromorphone to cause overdose or to be abused while still delivering therapeutic activity similar to that of the parent hydromorphone.

### RELATED U.S. APPLICATION DATA

This application claims priority under 35 U.S.C. §119(e) to U.S. provisional application Ser. No. 60/849,774 filed Oct. 6, 2006.

### FIELD OF INVENTION

The invention relates to pharmaceutical compounds, compositions and methods of using the same comprising a chemical moiety attached to hydromorphone. These inventions provide a variety of beneficial effects. Some inventions result in a substantial decrease in the potential of hydromorphone to cause overdose or to be abused. For instance, some inventions provide therapeutic activity similar to that of the parent hydromorphone when delivered at typical dosage ranges, however when delivered at higher doses the potential for overdose or abuse is reduced due to the limited bioavailability of hydromorphone as compared to hydromorphone delivered in an non-conjugated form. Alternatively or in addition, the prodrug may be designed to provide fast or slow release depending of its use for chronic pain versus acute pain. Additionally, some of the inventions may reduce the side effects associated with taking hydromorphone.

### BACKGROUND

Accidental and intentional overdose with prescription and over the counter drugs is a serious health problem with thousands of fatalities occurring each year as a result. Drug overdose is a significant and growing problem. It can occur accidentally, as when a child swallows pills without understanding the consequences, or intentionally as with suicide attempts. Emergency department reporting for a number of drugs rose substantially from 1994 to 2000. These include: amphetamines (10,118 to 18,555, up 83.4%), anticonvulsants, including carbamazepine (9,358 to 14,642, up 56.5%), muscle relaxants, including carisoprodol (12,223 to 19,001, up 55.5%), psychotherapeutic drugs, including SSRI antidepressants, tricyclic antidepressants, and other antidepressants (190,467 to 220,289, up 15.7%). Anxiolytics, sedatives, and hypnotics, including benzodiazepines (74,637 to 103,972, up 27.7%) and narcotic analgesics including codeine, hydrocodone, methadone, oxycodone, propoxyphene and others (44,518 to 99,317, up 123.1%).

Others have sought to prevent the potential harmful effects of overdose through various formulations. For example, opioids have been combined with antagonists in particular formulations designed to counteract the opioid if the formulation is disrupted before oral administration or is given parenterally. Extended release Concerta (methylphenidate) has been formulated in a paste to preclude administration by snorting or injection. Compositions have been coated with emetics in a quantity that if administered in moderation as intended no emesis occurs, however, if excessive amounts are consumed emesis is induced therefore preventing overdose. These methods, as well as conventional control release formulations, are often ineffective and circumvented.

Hydromorphone is a μ-opiod agonist represented by the following structure:

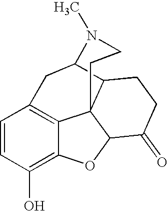

It is used to treat moderate to severe pain often in patients that have undergone surgery or have been severely injured. Trade names include Dilaudid®, Dilaudid-5, Palladone®, Palladone® SR, and Hydrostat IR. It may be administered orally, intravenously, intramuscularly, or rectally. It is also known as one of the most potent of all of the prescription narcotics when taken parenterally. For this reason, it is highly susceptible to abuse. Like other opioids, hydromorphone effects the central nervous system and gastrointestinal tract providing analgesia, drowsiness, mental clouding, mood changes, euphoria, dysphoria, respiratory depression, cough suppression, decreased gastrointestinal motility, nausea, vomiting, increased cerebrospinal pressure, increased biliary pressure, pinpoint constriction of the pupils, increased parasympathetic activity, and transient hyperglycemia.

Consequently, improved methods are needed to make pharmaceutically effective hydromorphone compounds, compositions and methods of using the same with reduced potential for overdose and/or resistance to manipulation while still providing necessary analgesia for various types of pain. Preferably, absorption of the composition into the brain is prevented or substantially diminished and/or delayed when delivered by routes other than oral administration.

### BRIEF DESCRIPTION OF DRAWINGS

FIG. 1 depicts the numbering scheme for hydromorphone.

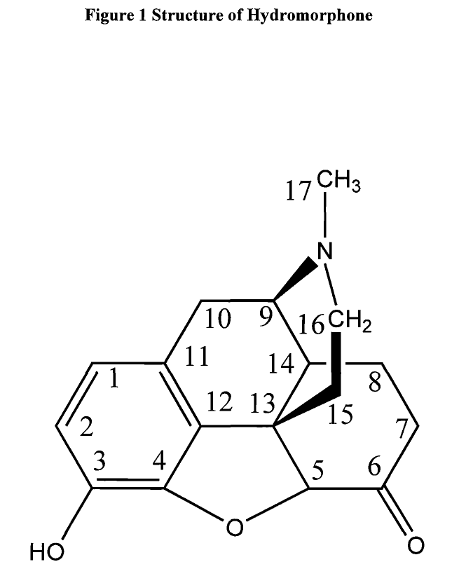

FIG. 2 depicts hydromorphone conjugated at the 3 position.

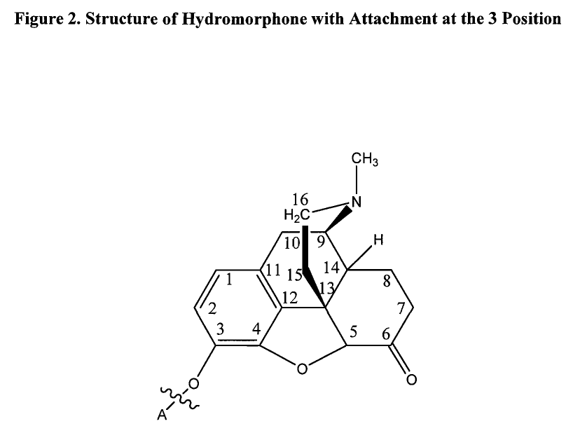

FIG. 3 depicts hydromorphone conjugated at the 3 and 6 positions.

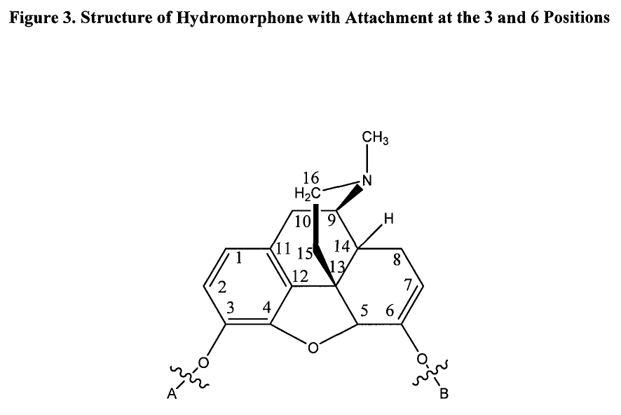

FIG. 4 depicts hydromorphone conjugated at the 6 position.

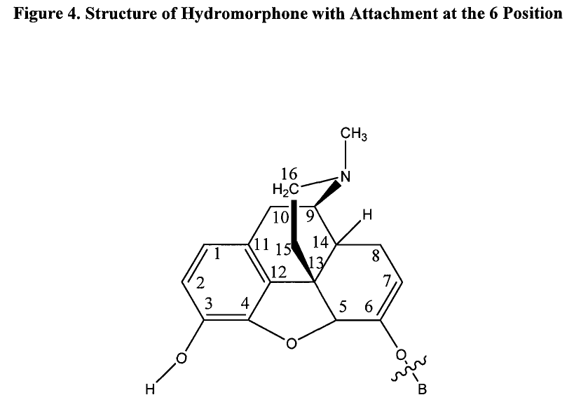

### DETAILED DESCRIPTION OF THE INVENTION

The invention relates to changing the pharmacokinetic and pharmacological properties of hydromorphone through covalent modification. Covalent attachment of a chemical moiety to hydromorphone may change one or more of the following: the rate of absorption, the extent of absorption, the metabolism, the distribution, and the elimination (ADME pharmacokinetic properties) of hydromorphone. As such, the alteration of one or more of these characteristics may be designed to provide fast or slow release depending of its use for chronic pain versus acute pain. Additionally, alteration of one or more of these characteristics may reduce the side effects associated with taking hydromorphone.

One aspect of the invention includes hydromorphone conjugates that when administered at a normal therapeutic dose the bioavailability (area under the time-versus-concentration curve; AUC) of hydromorphone provides a pharmaceutically effective amount of hydromorphone. As the dose is increased, however, the bioavailability of the covalently modified hydromorphone relative to the parent hydromorphone begins to decline, particularly for oral dosage forms. At suprapharmacological doses the bioavailability of the hydromorphone conjugate is substantially decreased as compared to the parent hydromorphone. The relative decrease in bioavailability at higher doses decreases or reduces the euphoria obtained when doses of the hydromorphone conjugate are taken above those of the intended prescription. This in turn diminishes the abuse potential, whether unintended or intentionally sought.

The invention provides hydromorphone prodrugs comprising hydromorphone covalently bound to a chemical moiety. The hydromorphone prodrugs can also be characterized as conjugates in that they possess a covalent attachment. They may also be characterized as conditionally bioreversible derivatives (“CBDs”).

In one embodiment, the hydromorphone prodrug (a compound of one of the formulas described herein) may exhibit one or more of the following advantages over free hydromorphones. The hydromorphone prodrug may prevent overdose by exhibiting a reduced pharmacological activity when administered at higher than therapeutic doses, e.g., higher than the prescribed dose. Yet when the hydromorphone prodrug is administered at therapeutic doses, the hydromorphone prodrug may retain similar pharmacological activity to that achieved by administering unbound hydromorphone. Also, the hydromorphone prodrug may prevent abuse by exhibiting stability under conditions likely to be employed by illicit chemists attempting to release the hydromorphone. The hydromorphone prodrug may prevent abuse by exhibiting reduced bioavailability when it is administered via parenteral routes, particularly the intravenous (“shooting”), intranasal (“snorting”), and/or inhalation (“smoking”) routes that are often employed in illicit use. Thus, the hydromorphone prodrug may reduce the euphoric effect associated with hydromorphone abuse. Thus, the hydromorphone prodrug may prevent and/or reduce the potential of abuse and/or overdose when the hydromorphone prodrug is used in a manner inconsistent with the manufacturer's instructions, e.g., consuming the hydromorphone prodrug at a higher than therapeutic dose or via a non-oral route of administration.

Preferably, the hydromorphone prodrug provides a serum release curve that does not increase above hydromorphone's toxicity level when administered at higher than therapeutic doses. The hydromorphone prodrug may exhibit a reduced rate of hydromorphone absorption and/or an increased rate of clearance compared to the free hydromorphone. The hydromorphone prodrug may also exhibit a steady-state serum release curve. Preferably, the hydromorphone prodrug provides bioavailability but prevents Cₘₐₓ spiking or increased blood serum concentrations.

Hydromorphone may be bound to one or more chemical moieties, denominated X and Z. A chemical moiety can be any moiety that decreases the pharmacological activity of hydromorphone while bound to the chemical moiety as compared to unbound (free) hydromorphone. The attached chemical moiety can be either naturally occurring or synthetic. In one embodiment, the invention provides an hydromorphone prodrug of Formula IA or IB:

H—Xₙ-Zₘ (IA)

H-Zₘ-Xₙ (IB)

wherein H is hydromorphone;
each X is independently a chemical moiety;
each Z is independently a chemical moiety that acts as an adjuvant and is different from at least one X;
n is an increment from 1 to 50, preferably 1 to 10; and
m is an increment from 0 to 50, preferably 0.
When m is 0, the hydromorphone prodrug is a compound of Formula (II):

H—Xₙ (II)

wherein each X is independently a chemical moiety.

Formula (II) can also be written to designate the chemical moiety that is physically attached to the hydromorphone:

H—X₁—(X)ₙ₋₁ (III)

wherein H is hydromorphone; X₁ is a chemical moiety, preferably a single amino acid; each X is independently a chemical moiety that is the same as or different from X₁; and n is an increment from 1 to 50.

H is hydromorphone and has the structure (IV), (V), or (VI) wherein A and B represent possible attachment sites for X.

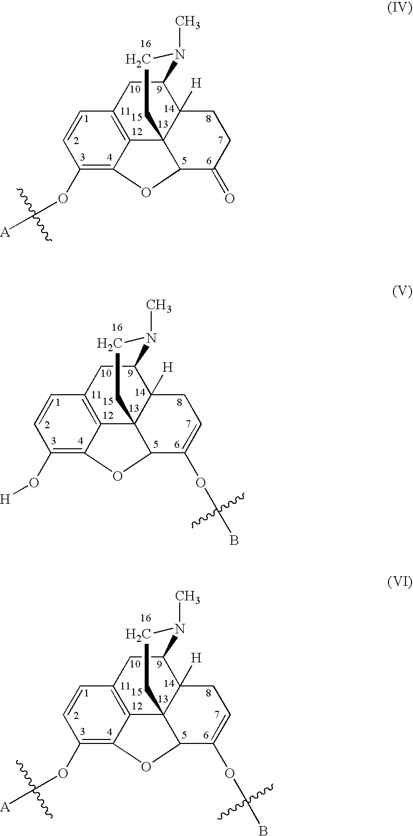

In an alternative embodiment, the N position of hydromorphone may be substituted with a chemical moiety with or without the presence of a linker. See U.S. Pat. No. 5,610,283 for methods of substituting opioids at the N position. Chemical moieties include, but are not limited to any of the carrier peptides listed below in Table 1.

Compounds, compositions and methods of the invention provide reduced potential for overdose, reduced potential for abuse or addiction and/or improve hydromorphone's characteristics with regard to high toxicities or suboptimal release profiles. Without wishing to be limited to the below theory, we believe that in some instances overdose protection results from a natural gating mechanism at the site of hydrolysis that limits the release of hydromorphone from the prodrug at greater than therapeutically prescribed amounts. Therefore, abuse resistance is provided by limiting the “rush” or “high” available from the hydromorphone released by the prodrug and limiting the effectiveness of alternative routes of administration for certain chemical moieties.

The invention utilizes covalent modification of hydromorphone to alter its ADME for certain delivery routes, e.g. routes other than oral, to decrease its potential for causing overdose or being abused. The hydromorphone is covalently modified in a manner that decreases its pharmacological activity, as compared to the unmodified hydromorphone, at doses above those considered therapeutic, e.g., at doses inconsistent with the manufacturer's instructions. When given at lower doses, such as those intended for therapy, covalently modified hydromorphone retains effective pharmacological activity. The covalent modification of hydromorphone may comprise the attachment of any chemical moiety through conventional chemistry. Preferably the chemical moiety is a carrier peptide.

Further, at times the invention is described as being hydromorphone attached to a peptide wherein the peptide is an amino acid, a dipeptide, a tripeptide, tetrapeptide, pentapeptide, or hexapeptide. Preferred lengths of the conjugates and other preferred embodiments are described herein. Preferred carriers are listed in Tables 1 and 2.

Persons that abuse prescription drugs commonly seek to increase their euphoria by snorting or injecting the drugs. These routes of administration increase the rate and extent of drug absorption and provide a faster, nearly instantaneous, effect. This increases the amount of drug that reaches the central nervous system where it has its effect. In a particular embodiment of the invention the bioavailability of the covalently modified hydromorphone is substantially decreased when taken by the intranasal and intravenous routes as compared to the parent hydromorphone. Thus the illicit practice of snorting and shooting the drug loses its advantage, i.e., the central nervous system effects are diminished.

In another embodiment of the invention, the solubility and dissolution rate of the composition is substantially changed under physiological conditions encountered in the intestine, at mucosal surfaces, or in the bloodstream. In another embodiment the solubility and dissolution rate substantially decrease the bioavailability of the hydromorphone prodrug, particularly at doses above those intended for therapy. In another embodiment the decrease in bioavailability occurs upon oral administration. In another embodiment the decrease in bioavailability occurs upon intranasal administration. In another embodiment the decrease in bioavailability occurs upon intravenous administration.

Another particular embodiment of the invention provides that when the covalently modified hydromorphone is provided in oral dosage form (e.g., a tablet, capsule, caplet, liquid dispersion, etc.) it has increased resistance to manipulation. For, instance, crushing of a tablet or disruption of a capsule does not substantially increase the rate and amount of hydromorphone absorbed when compositions of the invention are ingested.

Another embodiment of the invention provides compositions and methods of providing analgesia comprising administering to a patient compounds or compositions of the invention. Another embodiment provides a composition or method for treating pain in a patient i.e., acute and chronic pain. It should be noted that different conjugates maybe utilized to treat acute versus chronic pain.

Hydromorphone may be attached to the carrier peptide through the C-terminus, N-terminus, or side chain of the carrier peptide. Preferably, hydromorphone is attached to the C-terminus of the carrier peptide. It is preferred that aside from attachment of the carrier peptide to the hydromorphone neither is further substituted or protected. In one embodiment, the chemical moiety has one or more free carboxy and/or amine terminal and/or side chain group other than the point of attachment to the hydromorphone. The chemical moiety can be in such a free state, or an ester or salt thereof.

Another embodiment of the invention is a composition or method for safely delivering hydromorphone comprising providing a therapeutically effective amount of said hydromorphone which has been covalently bound to a chemical moiety wherein said chemical moiety reduces the rate of absorption of the hydromorphone as compared to delivering the unbound hydromorphone.

Another embodiment of the invention is a composition or method for reducing drug toxicity comprising providing a patient with hydromorphone which has been covalently bound to a chemical moiety wherein said chemical moiety increases the rate of clearance of hydromorphone when given at doses exceeding those within the therapeutic range of said hydromorphone.

Another embodiment provides a composition or method of reducing drug toxicity comprising providing a patient with hydromorphone which has been covalently bound to a chemical moiety wherein the chemical moiety provides a serum release curve which does not increase above the toxicity level of hydromorphone when given at doses exceeding those within the therapeutic range for unbound hydromorphone.

Another embodiment provides a composition that reduces or eliminates the toxic range of the Lethal Dose, 50% (LD₅₀) comprising providing a composition containing hydromorphone, which has been covalently bound to a chemical moiety.

Another embodiment of the invention is a composition or method for a sustained-release hydromorphone composition comprising providing hydromorphone which has been covalently bound to a chemical moiety, wherein said chemical moiety provides release of hydromorphone at a rate where the level of hydromorphone is within the therapeutic range but below toxic levels over an extended periods of time, e.g., 8-24 hours or greater.

Another embodiment of the invention is a composition or method for reducing bioavailability or preventing a toxic release profile of hydromorphone comprising hydromorphone covalently bound to a chemical moiety wherein said bound hydromorphone maintains a steady-state serum release curve which provides a therapeutically effective bioavailability but prevents spiking or increase blood serum concentrations compared to unbound hydromorphone when given at doses exceeding those within the therapeutic range of said hydromorphone.

Another embodiment of the invention is a composition or method for preventing a Cₘₐₓ spike for hydromorphone while still providing a therapeutically effective bioavailability curve comprising hydromorphone which has been covalently bound to a chemical moiety.

In another embodiment the compositions have substantially lower toxicity compared to unbound hydromorphone. In another embodiment the compositions reduce or eliminate the possibility of overdose by oral administration. In another embodiment the compositions reduce or eliminate the possibility of overdose by intranasal administration. In another embodiment the compositions reduce or eliminate the possibility of overdose by injection.

The invention further provides compositions or methods for altering hydromorphone in a manner that decreases their potential for abuse. Compositions and methods of the invention provide various ways to regulate pharmaceutical dosage through covalent attachment of hydromorphone to different chemical moieties. One embodiment provides a method of preventing overdose comprising administering to an individual hydromorphone which has been covalently bound to a chemical moiety.

Another embodiment of the invention is a method for reducing or preventing abuse or euphoric effect of a pharmaceutical composition, comprising providing, administering, or prescribing said composition to a human in need thereof, wherein said composition comprises a chemical moiety covalently attached to hydromorphone such that the pharmacological activity of hydromorphone is substantially decreased when the composition is used in a manner inconsistent with the manufacturer's instructions or in a manner that substantially increases the potential of overdose from hydromorphone.

Another embodiment of the invention is a method for reducing or preventing abuse or euphoric effect of a pharmaceutical composition, comprising consuming said composition, wherein said composition comprises a chemical moiety covalently attached to hydromorphone such that the pharmacological activity of hydromorphone is substantially decreased when the composition is used in a manner inconsistent with the manufacturer's instructions or in a manner that substantially decreases the potential of overdose from hydromorphone.

Another embodiment of the invention is any of the preceding methods wherein said pharmaceutical composition is adapted for oral administration, and wherein said hydromorphone is resistant to release from said chemical moiety when the composition is administered parenterally, such as intranasally or intravenously. Preferably, said hydromorphone may be released from said chemical moiety in the presence of acid and/or enzymes present in the stomach, intestinal tract, or blood serum.

Another embodiment of the invention is any of the herein described methods wherein said composition yields a therapeutic effect without substantial euphoria. Preferably, said hydromorphone provides a therapeutically bioequivalent AUC when compared to hydromorphone alone but does not provide a Cₘₐₓ which results in euphoria.

Another embodiment of the invention is a method for reducing or preventing abuse of a pharmaceutical composition, comprising orally administering said composition to a human in need thereof, wherein said composition comprises an amino acid or peptide covalently attached to hydromorphone such that the pharmacological activity of hydromorphone is substantially decreased when the composition is used in a manner inconsistent with the manufacturer's instructions.

Another embodiment is a method of preventing overdose of a pharmaceutical composition, comprising orally administering said pharmaceutical composition to a human in need thereof, wherein said composition comprises a carrier peptide covalently attached to hydromorphone in a manner that substantially decreases the potential of hydromorphone to result in overdose.

Another embodiment is a method for reducing or preventing the euphoric effect of a pharmaceutical composition, comprising orally administering said composition to a human in need thereof, wherein said composition comprises a carrier peptide covalently attached to hydromorphone such that the pharmacological activity of hydromorphone is substantially decreased when the composition is used in a manner inconsistent with the manufacturer's instructions.

For each of the recited methods of the invention the following properties may be achieved through bonding hydromorphone to the chemical moiety. In one embodiment, the toxicity of the compound may be substantially lower than that of the hydromorphone when delivered in its unbound state or as a salt thereof. In another embodiment, the possibility of overdose by oral administration is reduced or eliminated. In another embodiment, the possibility of overdose by intranasal administration is reduced or eliminated. In another embodiment, the possibility of overdose by injection administration is reduced or eliminated.

Another embodiment of the invention is wherein said attachment comprises an ester or carbonate bond. Another embodiment of the invention is wherein said hydromorphone covalently attaches to a chemical moiety through a ketone and/or hydroxyl.

The compositions and methods of the invention provide hydromorphone, which when bound to the chemical moiety provide safer and/or more effective dosages for hydromorphone through improved bioavailability curves and/or safer Cₘₐₓ and/or reduce area under the curve for bioavailability, particularly for abused substances taken in doses above therapeutic levels. As a result, the compositions and methods of the invention may provide improved methods of treatment for analgesia.

Preferably, the hydromorphone prodrug exhibits an oral bioavailability of hydromorphone of at least about 60% AUC (area under the curve), more preferably at least about 70%, 80%, 90%, 95%, 96%, 97%, 98%, 99%, compared to unbound hydromorphone. Preferably, the hydromorphone prodrug exhibits a parenteral bioavailability, e.g., intranasal, bioavailability of less than about 70% AUC, more preferably less than about 50%, 30%, 20%, 15%, 10%, 5%, 4%, 3%, 2%, 1%, compared to unbound hydromorphone.

In one embodiment, the hydromorphone prodrug provides pharmacological parameters (AUC, Cₘₐₓ, Tₘₐₓ, Cₘᵢₙ, and/or t_{1/2}) within 80% to 125%, 80% to 120%, 85% to 125%, 90% to 110%, or increments therein of unbound hydromorphone. It should be recognized that the ranges can, but need not be symmetrical, e.g., 85% to 105%.

In another embodiment, the toxicity of the hydromorphone prodrug is substantially lower than that of the unbound hydromorphone. For example, in a preferred embodiment, the acute toxicity is 1-fold, 2-fold, 3-fold, 4-fold, 5-fold, 6-fold, 7-fold, 8-fold, 9-fold, 10-fold less, or increments therein less lethal than oral administration of unbound hydromorphone.

For each of the described embodiments one or more characteristics as described throughout the specification may be realized. It should also be recognized that the compounds and compositions described throughout the specification may be utilized for a variety of novel methods of treatment, reduction of abuse potential, reduction of toxicity, improved release profiles, etc. An embodiment may obtain, one or more of: a conjugate with toxicity of hydromorphone that is substantially lower than that of unbound hydromorphone; a conjugate where the covalently bound chemical moiety reduces or eliminates the possibility of overdose by oral administration; a conjugate where the covalently bound chemical moiety reduces or eliminates the possibility of overdose by intranasal administration; and/or a conjugate where the covalently bound chemical moiety reduces or eliminates the possibility of overdose by injection.

In accordance with the invention and as used herein, the following terms are defined with the following meanings, unless explicitly stated otherwise.

The compounds, compositions and methods of the invention utilize “hydromorphone conjugates,” which are also referred to as hydromorphone prodrugs.

Throughout this application the use of “chemical moiety”—sometimes referred to as the “conjugate” or the “carrier”—is meant to include any chemical substance, naturally occurring or synthetic that decreases the pharmacological activity until the hydromorphone is released including at least carrier peptides, glycopeptides, carbohydrates, lipids, nucleic acids, nucleosides, or vitamins. Preferably, the chemical moiety is generally recognized as safe (“GRAS”).

Throughout this application the use of “carrier peptide” is meant to include naturally occurring amino acids, synthetic amino acids, and combinations thereof. In particular, carrier peptide is meant to include at least a single amino acid, a dipeptide, a tripeptide, an oligopeptide, a polypeptide, or the nucleic acid-amino acids peptides. The carrier peptide can comprise a homopolymer or heteropolymer of naturally occurring or synthetic amino acids.

The use of the term “straight carrier peptide” is meant to include amino acids that are linked via a —C(O)—NH— linkage, also referred to herein as a “peptide bond,” but may be substituted along the side chains of the carrier peptide. Amino acids that are not joined together via a peptide bond or are not exclusively joined through peptide bonds are not meant to fall within the definition of straight carrier peptide.

The use of the term “unsubstituted carrier peptide” is meant to include amino acids that are linked via a —C(O)—NH— linkage, and are not otherwise substituted along the side chains of the carrier peptide. Amino acids that are not joined together via a peptide bond or are not exclusively joined through peptide bonds are not meant to fall within the definition of unsubstituted carrier peptide.

“Peptide” is meant to include from 1 to 50 amino acids.

“Oligopeptide” is meant to include from 2 amino acids to 10 amino acids. “Polypeptides” are meant to include from 2 to 50 amino acids.

“Carbohydrates” includes sugars, starches, cellulose, and related compounds. More specific examples include for instance, fructose, glucose, lactose, maltose, sucrose, glyceraldehyde, dihydroxyacetone, erythrose, ribose, ribulose, xylulose, galactose, mannose, sedoheptulose, neuraminic acid, dextrin, and glycogen.

A “glycoprotein” is a compound containing carbohydrate (or glycan) covalently linked to protein. The carbohydrate may be in the form of a monosaccharide, disaccharide(s), oligosaccharide(s), polysaccharide(s), or their derivatives (e.g. sulfo- or phospho-substituted).

A “glycopeptide” is a compound consisting of carbohydrate linked to an oligopeptide composed of L- and/or D-amino acids. A glyco-amino-acid is a saccharide attached to a single amino acid by any kind of covalent bond. A glycosyl-amino-acid is a compound consisting of saccharide linked through a glycosyl linkage (O—, N— or S—) to an amino acid.

The “carrier range” or “carrier size” is determined based on the effect desired. It is preferably between one to 12 chemical moieties with one to 8 moieties being preferred. In another embodiment the number of chemical moieties attached is a specific number e.g., 1, 2, 3, 4, 5, 6, 7, 8, 9, or 10, etc. Alternatively, the chemical moiety may be described based on its molecular weight. It is preferred that the conjugate weight is below about 2,500 kD, more preferably below about 1,000 kD.

A “composition” as used herein, refers broadly to any composition containing a hydromorphone conjugate. A “pharmaceutical composition” refers to any composition containing a hydromorphone conjugate that only comprises components that are acceptable for pharmaceutical uses, e.g., excludes hydromorphone conjugates for immunological purposes.

Use of phrases such as “decreased”, “reduced”, “diminished”, or “lowered” includes at least a 10% change in pharmacological activity with respect to at least one ADME characteristic or at least one of AUC, Cₘₐₓ, Tₘₐₓ, Cₘᵢₙ, and t_{1/2} with greater percentage changes being preferred for reduction in abuse potential and overdose potential. For instance, the change may also be greater than 25%, 35%, 45%, 55%, 65%, 75%, 85%, 95%, 96%, 97%, 98%, 99%, or other increments.

Use of the phrase “similar pharmacological activity” means that two compounds exhibit curves that have substantially the same AUC, Cₘₐₓ, Tₘₐₓ, Cₘᵢₙ, and/or t_{1/2} parameters, preferably within about 30% of each other, more preferably within about 25%, 20%, 10%, 5%, 2%, 1%, or other increments.

“Cₘₐₓ” is defined as the maximum concentration of free hydromorphone in the body obtained during the dosing interval.

“Tₘₐₓ” is defined as the time to maximum concentration.

“Cₘᵢₙ” is defined as the minimum concentration of hydromorphone in the body after dosing.

“t_{1/2}” is defined as the time required for the amount of hydromorphone in the body to be reduced to one half of its value.

Throughout this application, the term “increment” is used to define a numerical value in varying degrees of precision, e.g., to the nearest 10, 1, 0.1, 0.01, etc. The increment can be rounded to any measurable degree of precision. For example, the range 1 to 100 or increments therein includes ranges such as 20 to 80, 5 to 50, 0.4 to 98, and 0.04 to 98.05.

“Acute pain” is defined as sharp or severe pain or discomfort that lasts for a short period of time. Preferably, a short period of time is less than 3 months for nociceptive or neurogenic pain, and less than 6 months for psychogenic pain.

“Chronic pain” is defined as moderate to severe pain that lasts for a long period of time. Preferably, a long period of time is more than 3 months for nociceptive or neurogenic pain and more than 6 months for psychogenic pain.

“Patient” as used herein, refers broadly to any animal that is in need of treatment, most preferably and animal that is in pain. The patient may be a clinical patient such as a human or a veterinary patient such as a companion, domesticated, livestock, exotic, or zoo animal. Animals may be mammals, reptiles, birds, amphibians, or invertebrates.

“Mammal” as used herein, refers broadly to any and all warm-blooded vertebrate animals of the class Mammalia, including humans, non-human primates, felines, canines, pigs, horses, sheep, etc.

“Pretreatment” as used herein, refers broadly to any and all preparation, treatment, or protocol that takes place before receiving a hydromorphone compound or composition of the invention.

“Treating” or “treatment” as used herein, refers broadly to preventing the disease, i.e., causing the clinical symptoms of the disease not to develop in a patient that may be exposed to or predisposed to the disease but does not yet experience or display symptoms of the disease, inhibiting the disease, i.e., arresting or reducing the development of the disease or its clinical symptoms, and/or relieving the disease, i.e., causing regression of the disease or its clinical symptoms. Treatment also encompasses an alleviation of signs and/or symptoms.

“Therapeutically effective amount” as used herein, refers broadly to the amount of a compound that, when administered to a patient for treating pain is sufficient to effect such treatment for pain. The “therapeutically effective amount” will vary depending on the compound, the disease and its severity and the age, weight, etc., of the patient to be treated. “Effective dosage” or “Effective amount” of the hydromorphone compound or composition is that which is necessary to treat or provide prophylaxis for hydromorphone.

“Selection of patients” and “Screening of patients” as used herein, refers broadly to the practice of selecting appropriate patients to receive the treatments described herein. Various factors including but not limited to age, weight, heath history, medications, surgeries, injuries, conditions, illnesses, diseases, infections, gender, ethnicity, genetic markers, polymorphisms, skin color, and sensitivity to hydromorphone treatment. Still other factors include those used by physicians to determine if a patient is appropriate to receive the treatments described herein.

“Diagnosis” as used herein, refers broadly to the practice of testing, assessing, assaying, and determining whether or not a patient is in pain.

Regarding stereochemistry, this patent is meant to cover all compounds discussed regardless of absolute configurations. Thus, natural, L-amino acids are discussed but the use of D-amino acids are also included.

For each of the embodiments recited herein, the carrier peptide may comprise of one or more of the naturally occurring (L-) amino acids: alanine, arginine, asparagine, aspartic acid, cysteine, glycine, glutamic acid, glutamine, histidine, isoleucine, leucine, lysine, methionine, proline, phenylalanine, serine, tryptophan, threonine, tyrosine, and valine. Other preferred amino acids include beta-alanine, beta-leucine, and tertiary leucine. In another embodiment the amino acid or peptide is comprised of one or more of the D-form of the naturally occurring amino acids. In another embodiment the amino acid or peptide is comprised of one or more unnatural, non-standard or synthetic amino acids such as, aminohexanoic acid, biphenylalanine, cyclohexylalanine, cyclohexylglycine, diethylglycine, dipropylglycine, 2,3-diaminoproprionic acid, homophenylalanine, homoserine, homotyrosine, naphthylalanine, norleucine, ornithine, pheylalanine(4-fluoro), phenylalanine(2,3,4,5,6 pentafluoro), phenylalanine(4-nitro), phenylglycine, pipecolic acid, sarcosine, tetrahydroisoquinoline-3-carboxylic acid, and tert-leucine. In another embodiment the amino acid or peptide comprises of one or more amino acid alcohols. In another embodiment the amino acid or peptide comprises of one or more N-methyl amino acids.

In another embodiment, the specific carriers listed in the table may have one or more of amino acids substituted with one of the 20 naturally occurring amino acids. It is preferred that the substitution be with an amino acid which is similar in structure or charge compared to the amino acid in the sequence. For instance, isoleucine (Ile)[I] is structurally very similar to leucine (Leu)[L], whereas, tyrosine (Tyr)[Y] is similar to phenylalanine (Phe)[F], whereas serine (Ser)[S] is similar to threonine (Thr)[T], whereas cysteine (Cys)[C] is similar to methionine (Met)[M], whereas alanine (Ala)[A] is similar to valine (Val)[V], whereas lysine (Lys)[K] is similar to arginine (Arg)[R], whereas asparagine (Asn)[N] is similar to glutamine (Gln)[Q], whereas aspartic acid (Asp)[D] is similar to glutamic acid (Glu)[E], whereas histidine (His)[H] is similar to proline (Pro)[P], and glycine (Gly)[G] is similar to tryptophan (Trp)[W]. In the alternative the preferred amino acid substitutions may be selected according to hydrophilic properties (i.e., polarity) or other common characteristics associated with the 20 essential amino acids. While preferred embodiments utilize the 20 natural amino acids for their GRAS characteristics, it is recognized that minor substitutions along the amino acid chain that do not affect the essential characteristics of the amino are also contemplated.

The hydromorphone conjugate may also be in salt form. Pharmaceutically acceptable salts, e.g., non-toxic, inorganic and organic acid addition salts, are known in the art. Exemplary salts include, but are not limited to, 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, bisulfate, bitartrate, borate, butyrate, calcium edetate, camphorate, camphorsulfonate, citrate, clavulariate, cyclopentanepropionate, digluconate, dodecylsulfate, edetate, edisylate, estolate, esylate, ethanesulfonate, finnarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, isothionate, lactate, lactobionate, laurate, laurylsulphonate, malate, maleate, mandelate, methanesulfonate, mucate, naphthylate, napsylate, nicotinate, N-methylglucamine ammonium salt, oleate, palmitate, pamoate, pantothenate, pectinate, phosphate, phosphateldiphosphate, pivalate, polygalacturonate, propionate, p-toluenesulfonate, saccharate, salicylate, stearate, subacetate, succinate, sulfate, sulfosaliculate, suramate, tannate, tartrate, teoclate, tosylate, triethiodide, undecanoate, and valerate salts, and the like.

In the invention, hydromorphone may be covalently attached to the peptide via the ketone group and a linker. This linker may be a small linear or cyclic molecule containing 2-6 atoms with one or more heteroatoms (such as O, S, N) and one or more functional groups (such as amines, amides, alcohols or acids) or may be made up of a short chain of either amino acids or carbohydrates). For example, glucose would be suitable as a linker.

In yet another embodiment of the invention, linkers can be selected from the group of all chemical classes of compounds such that virtually any side chain of the peptide can be attached. The linker should have a functional pendant group, such as a carboxylate, an alcohol, thiol, oxime, hydraxone, hydrazide, or an amine group, to covalently attach to the carrier peptide. In one preferred embodiment, the alcohol group of hydromorphone is covalently attached to the N-terminus of the peptide via a linker. In another preferred embodiment the ketone group of hydromorphone is attached to a linker through the formation of a ketal and the linker has a pendant group that is attached to the carrier peptide.

Additionally information regarding the attachment of active agents such as hydromorphone to carriers may be found in U.S. Pat. No. 7,060,708 and/or PCT/US03/05524 (WO 03/079972 A1), and/or PCT/US03/05525 (WO 03/072046 A1) each of which is hereby incorporated by reference in its entirety.

In addition to the hydromorphone prodrug, the pharmaceutical compositions of the invention may further comprise one or more pharmaceutical additives. Pharmaceutical additives include a wide range of materials including, but not limited to diluents and bulking substances, binders and adhesives, lubricants, glidants, plasticizers, disintegrants, carrier solvents, buffers, colorants, flavorings, sweeteners, preservatives and stabilizers, adsorbents, and other pharmaceutical additives known in the art.

Lubricants include, but are not limited to, magnesium stearate, calcium stearate, zinc stearate, powdered stearic acid, glyceryl monostearate, glyceryl palmitostearate, glyceryl behenate, silica, magnesium silicate, colloidal silicon dioxide, titanium dioxide, sodium benzoate, sodium lauryl sulfate, sodium stearyl fumarate, hydrogenated vegetable oil, talc, polyethylene glycol, and mineral oil.

Surface agents for formulation include, but are not limited to, sodium lauryl sulfate, dioctyl sodium sulfosuccinate, triethanolamine, polyoxyethylene sorbitan, poloxalkol, and quarternary ammonium salts; excipients such as lactose, mannitol, glucose, fructose, xylose, galactose, sucrose, maltose, xylitol, sorbitol, chloride, sulfate and phosphate salts of potassium, sodium, and magnesium; gelling agents such as colloidal clays; thickening agents such as gum tragacanth or sodium alginate, effervescing mixtures; and wetting agents such as lecithin, polysorbates or laurylsulphates.

Colorants can be used to improve appearance or to help identify the pharmaceutical composition. See 21 C.F.R., Part 74. Exemplary colorants include D&C Red No. 28, D&C Yellow No. 10, FD&C Blue No. 1, FD&C Red No. 40, FD&C Green #3, FD&C Yellow No. 6, and edible inks.

In embodiments where the pharmaceutical composition is compacted into a solid dosage form, e.g., a tablet, a binder can help the ingredients hold together. Binders include, but are not limited to, sugars such as sucrose, lactose, and glucose; corn syrup; soy polysaccharide, gelatin; povidone (e.g., Kollidon®, Plasdone®); Pullulan; cellulose derivatives such as microcrystalline cellulose, hydroxypropylmethyl cellulose (e.g., Methocel®), hydroxypropyl cellulose (e.g., Klucel®), ethylcellulose, hydroxyethyl cellulose, carboxymethylcellulose sodium, and methylcellulose; acrylic and methacrylic acid co-polymers; carbomer (e.g., Carbopol®); polyvinylpolypyrrolidine, polyethylene glycol (Carbowax®); pharmaceutical glaze; alginates such as alginic acid and sodium alginate; gums such as acacia, guar gum, and arabic gums; tragacanth; dextrin and maltodextrin; milk derivatives such as whey; starches such as pregelatinized starch and starch paste; hydrogenated vegetable oil; and magnesium aluminum silicate, as well as other conventional binders known to persons skilled in the art. Exemplary non-limiting bulking substances include sugar, lactose, gelatin, starch, and silicon dioxide.

Glidants can improve the flowability of non-compacted solid dosage forms and can improve the accuracy of dosing. Glidants include, but are not limited to, colloidal silicon dioxide, fumed silicon dioxide, silica gel, talc, magnesium trisilicate, magnesium or calcium stearate, powdered cellulose, starch, and tribasic calcium phosphate.

Plasticizers include, but are not limited to, hydrophobic and/or hydrophilic plasticizers such as, diethyl phthalate, butyl phthalate, diethyl sebacate, dibutyl sebacate, triethyl citrate, acetyltriethyl citrate, acetyltributyl citrate, cronotic acid, propylene glycol, castor oil, triacetin, polyethylene glycol, propylene glycol, glycerin, and sorbitol. Plasticizers are particularly useful for pharmaceutical compositions containing a polymer and in soft capsules and film-coated tablets.

Flavorings improve palatability and may be particularly useful for chewable tablet or liquid dosage forms. Flavorings include, but are not limited to maltol, vanillin, ethyl vanillin, menthol, citric acid, fumaric acid, ethyl maltol, and tartaric acid. Sweeteners include, but are not limited to, sorbitol, saccharin, sodium saccharin, sucrose, aspartame, fructose, mannitol, and invert sugar.

Preservatives and/or stabilizers improving storagability include, but are not limited to, alcohol, sodium benzoate, butylated hydroxy toluene, butylated hydroxyanisole, and ethylenediamine tetraacetic acid.

Disintegrants can increase the dissolution rate of a pharmaceutical composition. Disintegrants include, but are not limited to, alginates such as alginic acid and sodium alginate, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), polyvinylpolypyrrolidine (Plasone-XL®), guar gum, magnesium aluminum silicate, methyl cellulose, microcrystalline cellulose, polacrilin potassium, powdered cellulose, starch, pregelatinized starch, sodium starch glycolate (e.g., Explotab®, Primogel®).

Diluents increase the bulk of a dosage form and may make the dosage form easier to handle. Exemplary diluents include, but are not limited to, lactose, dextrose, saccharose, cellulose, starch, and calcium phosphate for solid dosage forms, e.g., tablets and capsules; olive oil and ethyl oleate for soft capsules; water and vegetable oil for liquid dosage forms, e.g., suspensions and emulsions. Additional suitable diluents include, but are not limited to, sucrose, dextrates, dextrin, maltodextrin, microcrystalline cellulose (e.g., Avicel®), microfine cellulose, powdered cellulose, pregelatinized starch (e.g., Starch 1500®), calcium phosphate dihydrate, soy polysaccharide (e.g., Emcosoy®), gelatin, silicon dioxide, calcium sulfate, calcium carbonate, magnesium carbonate, magnesium oxide, sorbitol, mannitol, kaolin, polymethacrylates (e.g., Eudragit®), potassium chloride, sodium chloride, and talc.

In embodiments where the pharmaceutical composition is formulated for a liquid dosage form, the pharmaceutical composition may include one or more solvents. Suitable solvents include, but are not limited to, water; alcohols such as ethanol and isopropyl alcohol; vegetable oil; polyethylene glycol; propylene glycol; and glycerin or mixing and combination thereof.

The pharmaceutical composition can comprise a buffer. Buffers include, but are not limited to, lactic acid, citric acid, acetic acid, sodium lactate, sodium citrate, and sodium acetate.

Hydrophilic polymers suitable for use in the sustained release formulation include: one or more natural or partially or totally synthetic hydrophilic gums such as acacia, gum tragacanth, locust bean gum, guar gum, or karaya gum, modified cellulosic substances such as methylcellulose, hydroxomethylcellulose, hydroxypropyl methylcellulose, hydroxypropyl cellulose, hydroxyethylcellulose, carboxymethylcellulose; proteinaceous substances such as agar, pectin, carrageen, and alginates; and other hydrophilic polymers such as carboxypolymethylene, gelatin, casein, zein, bentonite, magnesium aluminum silicate, polysaccharides, modified starch derivatives, and other hydrophilic polymers known to those of skill in the art or a combination of such polymers.

One of ordinary skill in the art would recognize a variety of structures, such as bead constructions and coatings, useful for achieving particular release profiles. It is also possible for the dosage form to combine any forms of release known to persons of ordinary skill in the art. These include immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is known in the art. See, e.g., U.S. Pat. No. 6,913,768.

However, it should be noted that the hydromorphone conjugate controls the release of hydromorphone into the digestive tract over an extended period of time resulting in an improved profile when compared to immediate release combinations and reduces and/or prevents abuse without the addition of the above additives. In a preferred embodiment no further sustained release additives are required to achieve a blunted or reduced pharmacokinetic curve (e.g. reduced euphoric effect) while achieving therapeutically effective amounts of hydromorphone release.

The dose range for adult human beings will depend on a number of factors including the age, weight and condition of the patient and the administration route. Tablets and other forms of presentation provided in discrete units conveniently contain a daily dose, or an appropriate fraction thereof, of the hydromorphone conjugate. The dosage form can contain a dose of about 1 mg to about 500 mg, about 2.5 mg to about 250 mg, about 5 mg to about 100 mg, about 7.5 mg to about 75 mg, or increments therein. In a preferred embodiment, the dosage form contains 2 mg, 2.5 mg, 8 mg, 12 mg, 16 mg, or 32 mg of a hydromorphone prodrug.

Tablets and other dosage forms provided in discrete units can contain a daily dose, or an appropriate fraction thereof, of one or more hydromorphone prodrugs.

Compositions of the invention may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24-hour period. The doses may be uneven doses with regard to one another or with regard to the individual components at different administration times. Preferably, a single dose is administered once daily.

Likewise, the compositions of the invention may be provided in a blister pack or other such pharmaceutical package. Further, the compositions of the present inventive subject matter may further include or be accompanied by indicia allowing individuals to identify the compositions as products for a prescribed treatment. The indicia may further additionally include an indication of the above specified time periods for administering the compositions. For example the indicia may be time indicia indicating a specific or general time of day for administration of the composition, or the indicia may be a day indicia indicating a day of the week for administration of the composition. The blister pack or other combination package may also include a second pharmaceutical product.

The compounds of the invention can be administered by a variety of dosage forms. Any biologically acceptable dosage form known to persons of ordinary skill in the art, and combinations thereof, are contemplated. Examples of such dosage forms include, without limitation, chewable tablets, quick dissolve tablets, effervescent tablets, reconstitutable powders, elixirs, liquids, solutions, suspension in an aqueous liquid or a non-aqueous liquid, emulsions, tablets, syringes, multi-layer tablets, bi-layer tablets, capsules, soft gelatin capsules, hard gelatin capsules, caplets, lozenges, chewable lozenges, beads, powders, granules, particles, microparticles, dispersible granules, cachets and combinations thereof. Preferably, said composition may be in the form of any of the known varieties of tablets (e.g., chewable tablets, conventional tablets, film-coated tablets, compressed tablets), capsules, liquid dispersions for oral administration (e.g., syrups, emulsions, solutions or suspensions).

However, the most effective means for delivering the abuse-resistant hydromorphone compounds of the invention is orally, to permit maximum release of hydromorphone to provide therapeutic effectiveness and/or sustained release while maintaining abuse resistance. When delivered by the oral route hydromorphone is released into circulation, preferably over an extended period of time as compared to hydromorphone alone.

It is preferred that the hydromorphone conjugate be compact enough to allow for a reduction in overall administration size. The smaller size of the hydromorphone prodrug dosage forms promotes ease of swallowing.

For oral administration, fine powders or granules containing diluting, dispersing and/or surface-active agents may be presented in a draught, in water or a syrup, in capsules or sachets in the dry state, in a non-aqueous suspension wherein suspending agents may be included, or in a suspension in water or a syrup. Where desirable or necessary, flavoring, preserving, suspending, thickening or emulsifying agents can be included.

Accordingly, the invention also provides methods comprising providing, administering, prescribing, or consuming a hydromorphone prodrug. The invention also provides pharmaceutical compositions comprising a hydromorphone prodrug. The formulation of such a pharmaceutical composition can optionally enhance or achieve the desired release profile.

### EXAMPLES

Any feature of the above-describe embodiments can be used in combination with any other feature of the above-described embodiments.

In order to facilitate a more complete understanding of the invention, Examples are provided below. However, the scope of the invention is not limited to specific embodiments disclosed in these Examples, which are for purposes of illustration only.

Table 1 lists exemplary carrier peptides to which hydromorphone may be covalently bonded. In some embodiments, amino acids are bound to the side chain of another amino acid. For example, the lysine side chain can support branching at the nitrogen atom, either at one or two positions (i.e., an amino acid can replace one or both hydrogen atoms bound to the nitrogen of the lysine side chain). Particular branched peptides, in accordance with some embodiments of the invention, are given in Table 1. Branching is depicted by showing the side chain bound amino acid(s) parenthetically, next to the amino acid from which it is being branched. When two amino acids are shown to be branched from a lysine, there can either be one or two attachment points at the side chain (i.e., one attachment point for a dipeptide, or two attachment points for two single amino acids).

TABLE 1

List of Amino Acids and Peptides to which
Hydromorphone May be Covalently Bonded

| | | | |
|---|---|---|---|
| Ala | Glu-Val-Val | Phe-Ser-Val | Tyr-Tyr-Phe |
| Arg | Gly-Asp-Val | Phe-Thr-Val | Tyr-Tyr-Val |
| Asn | Gly-Gly-Cha | Phe-Tyr-Val | Tyr-Val-Val |
| Asp | Gly-Gly-hPhe | Pro-Asp-Val | Val-Asp-Val |
| Cys | Gly-Gly-Ile | Pro-Gly-Val | Val-Gln-Val |
| Gln | Gly-Gly-Leu | Pro-Ile-Ile | Val-Glu-Gly |
| Glu | Gly-Pro-Val | Pro-Ile-Val | Val-Glu-Leu |
| Gly | Gly-Ser-Val | Pro-Leu-Ile | Val-Glu-Val |
| His | Gly-Thr-Val | Pro-Lys-Val | Val-Gly-Glu |
| Ile | Gly-Val-Val | Pro-Phe-Ile | Val-Gly-Val |
| Leu | Gly-Gly-Nle | Pro-Phe-Val | Val-Phe-Val |
| Lys | Gly-Gly-Phe | Pro-Pro-Cha | Val-Pro-Tyr |
| Met | Gly-Gly-Val | Pro-Pro-Ile | Val-Pro-Val |
| Phe | Gly-Ile-Ile | Pro-Pro-Leu | Val-Thr-Val |
| Pro | Gly-Lys-Val | Pro-Pro-Nle | Val-Tyr-Asp |
| Ser | Ile-Asp-Val | Pro-Pro-Phe | Val-Tyr-Asp |
| β-Leu | Ile-Glu-Val | Pro-Pro-Val | Val-Tyr-Glu |
| Thr | Ile-Gly-Val | Pro-Pro-Val | Val-Tyr-Gly |
| t-Leu | Ile-Phe-Val | Pro-Ser-Val | Val-Tyr-Ile |
| Trp | Ile-Ser-Val | Pro-Thr-Val | Val-Tyr-Leu |
| Tyr | Ile-Thr-Val | Pro-Tyr-Val | Val-Tyr-Lys |
| Val | Ile-Tyr-Val | Pro-Tyr-Val | Val-Tyr-Phe |
| β-Ala | Leu-Asp-Val | Pro-Val-Val | Val-Tyr-Pro |
| Glu^{pyro}-Glu | Leu-Glu-Val | Ser-Asp-Val | Val-Tyr-Val |
| Tyr-β-Ala | Leu-Gly-Val | Ser-Glu-Val | Lys-Tyr-Val-Ile |
| | | | [SEQ ID NO: 1] |
| β-Ala-β-Ala | Leu-Leu-Ile | Ser-Gly-Val | Tyr-Pro-Val-Ile |
| | | | [SEQ ID NO: 2] |
| Asp-Asp-Cha | Leu-Lys-Val | Ser-Ile-Val | Acetyl-Glu-Glu-Pro-Pro-Ile |
| | | | [SEQ ID NO: 3] |
| Asp-Asp-Ile | Leu-Phe-Val | Ser-Leu-Val | Asp-Asp-Gly-Gly-Ile |
| | | | [SEQ ID NO: 4] |
| Asp-Asp-Nle | Leu-Pro-Ile | Ser-Lys-Val | Asp-Asp-Leu-Leu-Ile |
| | | | [SEQ ID NO: 5] |
| Asp-Asp-Phe | Leu-Pro-Val | Ser-Phe-Val | Asp-Asp-Leu-Leu-Ile |
| | | | [SEQ ID NO: 5] |
| Asp-Asp-Val | Leu-Thr-Val | Ser-Pro-Val | Asp-Asp-Pro-Pro-Ile |
| | | | [SEQ ID NO: 6] |
| Asp-d-Asp-Ile | Leu-Tyr-Val | Ser-Tyr-Val | Glu-Glu-Gly-Gly-Phe |
| | | | [SEQ ID NO: 7] |
| Asp-Glu-Val | Lys-Asp-Val | Ser-Val-Val | Glu-Glu-Leu-Leu-Leu |
| | | | [SEQ ID NO: 8] |
| Asp-Gly-Val | Lys-Glu-Val | Thr-Asp-Val | Glu-Glu-Phe-Phe-Leu |
| | | | [SEQ ID NO: 9] |
| Asp-Ile-Val | Lys-Gly-Val | Thr-Glu-Val | Glu-Glu-Phe-Pro-Ile |
| | | | [SEQ ID NO: 10] |
| Asp-Leu-Val | Lys-Ile-Val | Thr-Gly-Val | Glu-Glu-Pro-Pro-Leu |
| | | | [SEQ ID NO: 11] |
| Asp-Lys-Val | Lys-Leu-Val | Thr-Leu-Val | Glu-Glu-Pro-Phe-Ile |
| | | | [SEQ ID NO: 12] |
| Asp-Phe-Val | Lys-Lys-Ile | Thr-Lys-Val | Glu-Glu-Glu-Glu-Ile |
| | | | [SEQ ID NO: 13] |
| Asp-Pro-Val | Lys-Lys-Leu | Thr-Phe-Val | Glu-Glu-Phe-Phe-Phe |
| | | | [SEQ ID NO: 14] |
| Asp-Ser-Val | Lys-Lys-Val | Thr-Pro-Val | Gly-Gly-Glu-Glu-Ile |
| | | | [SEQ ID NO: 15] |
| Asp-Thr-Val | Lys-Phe-Val | Thr-Ser-Val | Lys-Lys-Leu-Leu-Ile |
| | | | [SEQ ID NO: 16] |
| Asp-Tyr-VaI | Lys-Pro-Val | Thr-Thr-Ile | Lys-Lys-Pro-Pro-Ile |
| | | | [SEQ ID NO: 17] |
| Asp-Val-Val | Lys-Thr-Val | Thr-Thr-Val | Phe-Phe-Glu-Glu-Ile |
| | | | [SEQ ID NO: 18] |
| Gln-Gln-Ile | Lys-Tyr-Val | Thr-Tyr-Val | Phe-Phe-Phe-Phe-Phe |
| | | | [SEQ ID NO: 19] |
| Gln-Gln-Val | Lys-Tyr-Val | Thr-Val-Val | Thr-Thr-Gly-Gly-Ile |
| | | | [SEQ ID NO: 20] |
| Gln-Gln-β-Ala | Lys-Val-Val | Tyr-Asp-Val | Thr-Thr-Phe-Phe-Ile |
| | | | [SEQ ID NO: 21] |
| Gln-Pro-Val | Phe-Asp-Val | Tyr-Glu-Val | Tyr-Tyr-Leu-Leu-Ile |
| | | | [SEQ ID NO: 22] |
| Glu-Glu-Cha | Phe-Glu-Val | Tyr-Gly-Val | Tyr-Tyr-Phe-Phe-Ile |
| | | | [SEQ ID NO: 23] |
| Glu-Glu-hPhe | Phe-Gly-Val | Tyr-Ile-Val | Tyr-Tyr-Pro-Pro-Ile |
| | | | [SEQ ID NO: 24] |
| Glu-Glu-Ile | Phe-Ile-Val | Tyr-Leu-Val | Tyr-Tyr-Pro-Phe-Ile |
| | | | [SEQ ID NO: 25] |
| Glu-Glu-Leu | Phe-Leu-Val | Tyr-Lys-Val | Tyr-Tyr-Phe-Phe-Ile |
| | | | [SEQ ID NO: 26] |
| Glu-Glu-Nle | Phe-Lys-Val | Tyr-Phe-Val | Tyr-Tyr-Phe-Phe-Val |
| | | | [SEQ ID NO: 27] |
| Glu-Glu-Phe | Phe-Phe-Cha | Tyr-Pro-Val | Asp-Asp-Lys(Asp₂) |
| Glu-Glu-Val | Phe-Phe-hPhe | Tyr-Ser-Val | Glu-Glu-Lys(Glu₂) |
| Glu-Gly-Val | Phe-Phe-Ile | Tyr-Thr-VaI | Phe-Phe-Lys(Phe₂) |
| Glu-Leu-Val | Phe-Phe-Leu | Tyr-Tyr-Ala | Pro-Pro-Lys(Pro₂) |
| Glu-Lys-Val | Phe-Phe-Nle | Tyr-Tyr-Cha | Tyr-Tyr-Lys(Tyr₂) |
| Glu-Phe-Val | Phe-Phe-Phe | Tyr-Tyr-hPhe | Ethyl Carbonate |
| Glu-Ser-Val | Phe-Phe-Val | Tyr-Tyr-Ile | galactose-Gly-Gly-Ile |
| Glu-Thr-Val | Phe-Phe-Val | Tyr-Tyr-Leu | galactose-Gly-Gly-Leu |
| Glu-Tyr-Val | Phe-Pro-Val | Tyr-Tyr-Nle | galactose-Ile |

The following Table lists preferred hydromorphone conjugates made according to the invention.

TABLE 2
List of Hydromorphone (HM) Conjugates attached through the 6
position to the C-terminus of the amino acid According to the
invention (for clarity purposes the amino acid that is next to
the -HM is the amino acid that is connected to the HM).

| | |
|---|---|
| YYFFI-HM | KKI-HM |
| EEFFF-HM | KKL-HM |
| YYI-HM | KKV-HM |
| YYL-HM | EEI-HM |
| YYV-HM | EEL-HM |
| GGI-HM | EEV-HM |
| GGL-HM | FFI-HM |
| GGV-HM | FFL-HM |
| PPI-HM | FFV-HM |
| PPL-HM | DDI-HM |
| PPV-HM | |

Hydromorphone conjugates also include the OAc and OEt derivatives of the above conjugates (at the 3 position).

### General Synthesis of Peptide Hydromorphone Conjugates

Peptide conjugates were synthesized by the general method described herein below with respect to the β-Alanine conjugate.

An iterative approach can be used to identify favorable conjugates by synthesizing and testing single amino acid conjugates, and then extending the peptide one amino acid at a time to yield dipeptide and tripeptide conjugates, etc. The parent single amino acid prodrug candidate may exhibit more or less desirable characteristics than its di- or tripeptide offspring candidates.

### General Synthesis of Single Amino Acid Hydromorphone Conjugates

### Procedure for Synthesis of β-Alanine-Hydromorphone Dihydrochloride

To a stirring solution of hydromorphone free base in dimethylforamide (DMF), imidazole and then tert-butyldimethylsilyl chloride were added, under argon at ambient temperature. The solution was allowed to stir for 6 hours and the reaction was quenched with water. The solvent was removed under reduced pressure and then the crude product dissolved in ethyl acetate and washed with brine solution, dried over sodium sulfate and condensed to afford HM-TBDMS.

Hydromorphone tert-butyldimethylsilyl ether was dissolved in tetrahydrofuran (THF) under argon at ambient temperature. The solution was cooled to 0° C. and then LiN(TMS)₂ was added and the solution was allowed to stir for 10 minutes. Boc-β-Ala-OSu was then added to the solution and the reaction was monitored by HPLC. The reaction was quenched by addition of NH₄Cl solution. The solvent was removed under reduced pressure, dissolved in ethyl acetate and washed with satd. NaHCO₃ solution, brine solution and dried over sodium sulfate. The solvent was removed under reduced pressure to afford Boc-β-Ala-HM-TBDMS. The crude product was purified by preparative HPLC.

To remove the TBDMS protecting group, Boc-β-Ala-HM-TBDMS was dissolved in a 0.2 M solution of NH₄F in methanol and allowed to stir for 8 hrs, under argon at ambient temperature. The solvent was removed under reduced pressure to afford Boc-β-Ala-HM. The material was purified by crystallization in methanol and tert-butyldimethyl ether.

Boc-β-Ala-HM was then dissolved in 4N HCl in dioxane, under argon at 0° C. and allowed to stir for 2 hours. The solvent was removed under reduced pressure to yield β-Ala-HM

It should be recognized that synthesis for the 6 position is applicable to the 3 position as well.

The Examples are further separated into categories based on the attachment of the carrier peptides to hydromorphone. Specifically, the first category relates to Examples that are directed to mono-substituted conjugates.

### A. Mono-Substituted Hydromorphone

Within the first category, in addition to the above, Examples showing substitution at the 6 position of hydromorphone with tripeptides and pentapeptides are provided. Again, it is possible to substitute at the 3 position of hydromorphone with a chemical moiety, but this is not a preferred site of substitution.

1. Attachment at the 6 Position of Hydromorphone

### Example 1

### Synthesis of Hydromorphone Bound to a Tripeptide at the 6 Position

To a solution of X—O⁶-hydromorphone.2HCl (1 mmol) in DMF were added NMM (10 mmol) and Boc-Z—Y-OSu (1.2 mmol). The reaction mixture was stirred at room temperature overnight. Solvent was evaporated to the residue was added saturated NaHCO₃ solution and stirred for 1 h. The precipitate was filtered, thoroughly washed with water and dried to give the title compound.

### Deprotection of Boc-Z—Y—X—O⁶-Hydromorphone:

Deprotection is performed in the same manner as the general method mentioned above to give Z—Y—X—O⁶-Hydromorphone.2HCl.

### Example 2

### Synthesis of Hydromorphone Bound to a Pentapeptide at the 6 Position

Procedure for Synthesis of Glu₂-Phe₃-Hydromorphone.Dihydrochloride

To a stirring solution of hydromorphone free base in DMF, imidazole and then tert-butyldimethylsilyl chloride were added, under argon at ambient temperature. The solution was allowed to stir for 6 hours and then the reaction was quenched with water and the solvent was removed under reduced pressure. The crude product was dissolved in ethyl acetate and washed with brine solution, dried over sodium sulfate and condensed to afford HM-TBDMS.

Hydromorphone tert-butyldimethylsilyl ether was dissolved in THF under argon at ambient temperature. The solution was cooled to 0° C. and then LiN(TMS)₂ was added and the solution was allowed to stir for 10 minutes. Boc-Phe-OSu was then added to the solution and the reaction was monitored by HPLC. The reaction was then quenched by addition of NH₄Cl solution. The solvent was removed under reduced pressure, dissolved in ethyl acetate and washed with a satd. NaHCO₃ solution, a brine solution, dried over sodium sulfate and the solvent was removed under reduced pressure to afford Boc-Phe-HM-TBDMS. The crude product was purified by preparative HPLC.

Boc-Phe-HM-TBDMS was dissolved in 4N HCl in dioxane, under argon at 0° C. and allowed to stir for 2 hours. The solvent was removed under reduced pressure to yield Phe-HM-TBDMS.

H-Glu(OtBu)-OMe HCl was dissolved in THF and then N-methylmorpholine (NMM) and Boc-Glu(OtBu)-OSu were added and the solution was allowed to stir under argon at ambient temperature for 18 hours. The solvent was removed under reduced pressure and the product was dissolved in ethyl acetate and washed with 3% AcOH solution, satd. NaHCO₃ solution, brine solution and dried over sodium sulfate. The solvent was removed under reduced pressure to afford Boc-(Glu(OtBu))₂-OMe.

Boc-(Glu(OtBu))₂-OMe was dissolved in THF at 0° C. A solution of LiOH.H₂O in water was added and allowed to stir for 3 hours. The reaction was quenched by addition of 3% acetic acid (pH 5.5). The product was extracted in isopropyl acetate, washed with brine solution, dried over sodium sulfate and the solvent removed under reduced pressure to afford Boc-(Glu(OtBu))₂-OH.

To purify the material, Boc-(Glu(OtBu))₂-OH was dissolved in acetonitrile upon heating. Dicyclohexylamine (DCHA) was then added to the solution and allowed to cool to ambient temperature. The precipitate was filtered off and washed with acetonitrile to afford Boc-(Glu(OtBu))₂-OH.DCHA.

Boc-(Glu(OtBu))₂-OH.DCHA was dissolved in ethyl acetate and a 5% KHSO₄ solution was added and allowed to stir at ambient temperature for 20 minutes. The organic layer was separated and the product again extracted from the aqueous phase with ethyl acetate. The organic extracts were combined and the solvent removed under reduced pressure to afford Boc-(Glu(OtBu))₂-OH.

Boc-Glu(OtBu)₂-OH was dissolved in THF and the solution was cooled to 0° C., under argon. NHS was added and the solution was allowed to stir for 10 minutes. Dicyclohexycarbodiimide (DCC) was then added and the solution was allowed to warm up to ambient temperature and stirred for 18 hours. The solid (DCU) was filtered off, washed with THF and the filtrate was condensed under reduced pressure to afford Boc-(Glu(OtBu))₂-OSu.

To a solution of H-Phe₂-OMe in THF, N-methylmorpholine was added and the solution was allowed to stir for 30 minutes under argon, at 10° C. Then the solution of Boc-(Glu(OtBu))₂-OSu in THF was added and the solution was allowed to stir for 4 hours under argon, at 10° C. The reaction was quenched with a 5% NaHCO₃ solution. The product extracted with isopropyl acetate, washed with brine solution, dried over sodium sulfate and the solvent removed under reduced pressure to afford Boc-(Glu(OtBu))₂-Phe₂-OMe.

Boc-(Glu(OtBu))₂-Phe₂-OMe was dissolved in THF at 0° C. A solution of LiOH.H₂O in water was added and allowed to stir for 3 hours. The reaction was quenched by addition of 3% acetic acid (pH 5.5). The product was extracted in isopropyl acetate, washed with brine solution, dried over sodium sulfate and the solvent removed under reduced pressure to afford Boc-(Glu(OtBu))₂-Phe₂-OH.

The coupling of Boc-(Glu(OtBu))₂-Phe₂-OH and Phe-HM-TBDMS was carried out by dissolving Boc-(Glu(OtBu))₂-Phe₂-OH in THF and adding NHS and then DCC to the solution. The solution was allowed to stir at ambient temperature and stirred for 18 hours. The solid (DCU) was filtered off, washed with THF and the filtrate added to a cold solution (0° C.) of Phe-HM-TBDMS in THF and NMM. The reaction was monitored by HPLC analysis and after 5 hours the reaction was quenched by addition of a 0.5% NaHCO₃ solution. The solution was allowed to stir for 10 minutes and then water was added to the mixture. The precipitate was filtered off, washed with water and dried, yielding Boc-(Glu(OtBu))₂-Phe₃-HM-TBDMS. The product was purified by preparative HPLC.

To remove the TBDMS protecting group, Boc-(Glu(OtBu))₂-Phe₃-HM-TBDMS was dissolved in a 0.2 M solution of NH₄F in methanol, under argon at ambient temperature. Once the reaction was completed the solvent was removed under reduced pressure to afford Boc-(Glu(OtBu))₂-Phe₃-HM. The material was purified by crystallization in methanol and tert-butyldimethyl ether.

Boc-(Glu(OtBu))₂-Phe₃-HM was then dissolved in 4N HCl in dioxane, under argon at 0° C. and allowed to stir for 2 hours. The solvent was removed under reduced pressure to yield Glu₂-Phe₃-HM.

### B. Di-Substituted Hydromorphone Conjugates

The second category of Examples relates to disubstituted hydromorphone conjugates at the 3 and 6 positions. The chemical moiety may be for instance two carrier peptides varied in both length and make-up or they may be identical.

1. Attachment at the 3 Position and 6 Position of Hydromorphone

### Example 3

### General Synthesis of Hydromorphone Bound to an Amino Acid at the 3 Position and at the 6 Position: [Boc-X]₂-Hydromorphone

To a solution of hydromorphone free base (2.04 g, 6.47 mmol) in THF (35 ml) was added LiN(TMS)₂ (19.41 ml, 19.41 mmol) and stirred for ˜30 mins. To this was added solid Boc-X-OSu (X=amino acid, 21 mmol) at one time and the reaction mixture was stirred at room temperature overnight. The solution was neutralized with 1N HCl and the THF was removed under reduced pressure. The residue was diluted with EtOAc (200 mL), satd. NaHCO₃ (150 mL) was added and stirred for 1 h. EtOAc part was washed with NaHCO3 and brine. Dried over Na₂SO₄ and evaporated to dryness. Compound was obtained by purification over silica gel column (30% EtOAc/Hexane).

### Deprotection of [Boc-X]₂-Hydromorphone:

General method of deprotection: The above compound was reacted with 4N HCl/dioxane (25 mL/gm) at room temperature for 4 h. Solvent was evaporated and dried over vacuum to give X₂-Hydromorphone.3HCl.

### Pharmacokinetic Data for Hydromorphone Conjugates

The invention is illustrated by pharmacokinetic studies with hydromorphone that has been covalently modified by attachment to various moieties such as specific short chained amino acid sequences including tri-, and pentapeptides. Studies include pharmacokinetic evaluations of the various drug conjugates administered by the oral and intranasal routes. Collectively the compounds demonstrate that active agents may be modified by covalent attachment to various moieties and retain their therapeutic value at normal doses while preventing potential overdose by oral administration and prevention of abuse through intranasal administration.

The Examples illustrate the applicability of attaching various moieties to hydromorphone to reduce the potential for overdose while maintaining therapeutic value. The invention is illustrated by pharmacokinetic studies with various peptide hydromorphone conjugates. The Examples illustrate the compounds and compositions for reducing the potential for overdose and abuse while maintaining therapeutic value wherein the active agent hydromorphone (HM) is covalently attached to a chemical moiety.

Oral, and intranasal bioavailability studies of hydromorphone and hydromorphone conjugates were conducted in male Sprague-Dawley rats. Doses of hydromorphone hydrochloride and hydromorphone conjugates containing equivalent amounts of hydromorphone were administered in deionized water. Oral administration was in 0.5 ml by gavage needle. Intranasal doses were administered by placing 20 microliters into the nasal flares of rats anesthetized with isoflurane. Plasma was collected by retroorbital sinus puncture under isoflurane anesthesia. hydromorphone were determined by LC/MS/MS.

### Example 4

### Decreased Oral Cₘₐₓ of Hydromorphone Conjugates

Male Sprague-Dawley rats were provided water ad libitum, fasted overnight and dosed by oral gavage with oxycodone conjugates or oxycodone HCl. All doses contained equivalent amounts of hydromorphone base. Plasma hydromorphone concentrations were measured by ELISA (102919, Neogen, Corporation, Lexington, Ky.) and/or LC/MS. These examples illustrate that doses of hydromorphone conjugates decrease the peak level (Cₘₐₓ) of hydromorphone as compared to that produced by equimolar (hydromorphone base) doses of hydromorphone HCl when given by the oral route of administration.

### Example 5

### Decreased Intranasal Bioavailability (AUC and Cₘₐₓ) of Hydromorphone Conjugates

Male Sprague-Dawley rats were provided water ad libitum and doses were administered by placing 0.02 ml of water containing hydromorphone conjugates or hydromorphone bitartrate into the nasal flares. All doses contained equivalent amounts of hydromorphone base. Plasma hydromorphone concentrations were measured by ELISA (hydromorphone, 102919, Neogen, Corporation, Lexington, Ky.) and/or LC/MS. The assay is specific for hydromorphone. These examples illustrate that hydromorphone conjugates decrease the peak level (Cₘₐₓ) and total absorption (AUC) of hydromorphone as compared to those produced by equimolar (hydromorphone base) doses of hydromorphone HCl when given by the intranasal route of administration.

The following Tables include Hydromorphone amino acid and peptide HCl salt conjugates that were synthesized and tested. Both oral and intranasal bioavailabilities were measured. The bold values are the “corrected values”, that were calculated based on the bioavailability of the control (HM).

TABLE 3
Oral Bioavailability of Hydromorphone Conjugates

| | | (Y2F2I- | | | | | | | | | | (Y2F2I- |
|---|---|---|---|---|---|---|---|---|---|---|---|---|
| | | HM- | | | | | | | | | | HM- |
| Oral | (Y2F2I) | OAc) | (D2I) | (Y2I) | (E2F3) | (G2L) | (E2L) | (G2V) | (K2V) | (P2V) | (Y2V) | OEt) |
| AUC | 74 | 54 | 46 | 52 | 128 | 41 | 51 | 86 | 78 | 40 | 53 | 149 |
| % | 40 | 29 | 25 | 32 | 79 | 22 | 27 | 53 | 43 | 24 | 30 | 71 |
| Cmax | 39 | 30 | 36 | 43 | 60 | 34 | 42 | 47 | 54 | 40 | 47 | 65 |
| % | 52 | 40 | 49 | 66 | 78 | 43 | 49 | 63 | 60 | 55 | 53 | 77 |

TABLE 4
Oral Bioavailability of Hydromorphone Tripeptide Conjugates

| | | | | | | (Y2I- | | | | | | |
|---|---|---|---|---|---|---|---|---|---|---|---|---|
| | | | | | | HM- | | | | | | |
| Oral | (E2I) | (F2I) | (G2I) | (K2I) | (P2I) | OEt) | (E2V) | (F2V) | (P2L) | (Y2L) | (F2L) | (K2L) |
| AUC | 94 | 34 | 30 | 39 | 35 | 24 | 36 | 13 | 39 | 21 | 18 | 44 |
| % | 56 | 18 | 17 | 24 | 21 | 14 | 21 | 8 | 22 | 12 | 11 | 24 |
| Cmax | 56 | 42 | 24 | 44 | 43 | 31 | 39 | 28 | 35 | 23 | 17 | 41 |
| % | 73 | 47 | 32 | 56 | 60 | 42 | 50 | 36 | 45 | 30 | 24 | 50 |

TABLE 5
Intranasal Bioavailability of Hydromorphone Conjugates

| | (Y2F2I- | (Y2F2I- | | | | | | | | | | |
|---|---|---|---|---|---|---|---|---|---|---|---|---|
| | HM- | HM- | | | | | | | | | | |
| IN | OAc) | OEt) | (Y2F2I) | (D2I) | (Y2I) | (P2L) | (Y2L) | (G2I) | (K2I) | (E2F3) | (G2L) | (E2L) |
| AUC | 51 | 48 | 28 | 387 | 619 | 352 | 491 | 147 | 421 | 384 | 357 | 575 |
| % | 59 | 56 | 4 | 55 | 88 | 53 | 74 | 24 | 68 | 52 | 52 | 84 |
| Cmax | 66 | 63 | 62 | 721 | 732 | 600 | 642 | 686 | 881 | 566 | 768 | 880 |
| % | 72 | 68 | 7 | 85 | 86 | 70 | 75 | 66 | 85 | 57 | 85 | 98 |

TABLE 6
Intranasal Bioavailability of Hydromorphone Tripeptide Conjugates

| IN | (E2I) | (F2I) | (P2I) | (Y2I-HM-OEt) | (E2V) | (G2V) | (K2V) | (P2V) | (Y2V) | (F2L) | (K2L) | (F2V) |
|---|---|---|---|---|---|---|---|---|---|---|---|---|
| AUC | 517 | 695 | 535 | 717 | 211 | 385 | 560 | 479 | 492 | 679 | 648 | 658 |
| % | 59 | 79 | 77 | 103 | 31 | 51 | 75 | 71 | 73 | 92 | 87 | 98 |
| Cmax | 875 | 916 | 888 | 845 | 629 | 760 | 931 | 938 | 686 | 945 | 949 | 920 |
| % | 88 | 92 | 95 | 90 | 70 | 73 | 89 | 101 | 73 | 96 | 96 | 103 |

TABLE 7
Bioavailability of Hydromorphone Conjugates.

| | | | | theor. |
|---|---|---|---|---|
| Compound Class | Compound | oral AUC | IN AUC | potency |
| pentapeptide | EEFFF-HM | 79 | 52 | 27 |
| tripeptide | DDI-HM | 25 | 55 | 40 |
| tripeptide | EEI-HM | 56 | 59 | 39 |
| tripeptide | FFI-HM | 18 | 79 | 37 |
| tripeptide | GGI-HM | 17 | 24 | 48 |
| tripeptide | KKI-HM | 24 | 68 | 35 |
| tripeptide | PPI-KM | 21 | 77 | 43 |
| tripeptide | YYI-HM | 32 | 88 | 28 |
| tripeptide | YYI-HM-OAc | 29 | 73 | 34 |
| tripeptide | YYI-HM-OEt | 14 | 103 | 33 |
| pentapeptide | YYFFI-HM | 40 | 4 | 26 |
| pentapeptide | YYFFI-HM-OAc | nd | 59 | 25 |
| pentapeptide | YYFFI-HM-OEt | 71 | 56 | 24 |
| tripeptide | EEL-HM | 27 | 84 | 39 |
| tripeptide | FFL-HM | 11 | 92 | 37 |
| tripeptide | GGL-HM | 22 | 52 | 49 |
| tripeptide | KKL-HM | 24 | 87 | 35 |
| tripeptide | PPL-HM | 22 | 53 | 43 |
| tripeptide | YYL-HM | 12 | 74 | 36 |
| tripeptide | EEV-HM | 21 | 31 | 40 |
| tripeptide | FFV-HM | 8 | 98 | 38 |
| tripeptide | GGV-HM | 53 | 51 | 50 |
| tripeptide | KKV-HM | 43 | 75 | 36 |
| tripeptide | PPV-HM | 24 | 71 | 44 |
| tripeptide | YYV-HM | 30 | 73 | 36 |

Collectively, the examples illustrate the application of the invention for reducing the overdose potential of hydromorphone. These examples establish that hydromorphone can be covalently modified by attachment of a chemical moiety in a manner that maintains therapeutic value over a normal dosing range, while substantially decreasing if not eliminating the possibility of overdose by oral or intranasal routes of administration with the hydromorphone.

As will be apparent to one skilled in the art, various modifications can be made within the scope of the invention description. Such modifications being within the ability of one skilled in the art form a part of the present invention and are embraced by the recitations in the claims set forth below and equivalents thereof.

1. A compound of the formula I or the formula II: 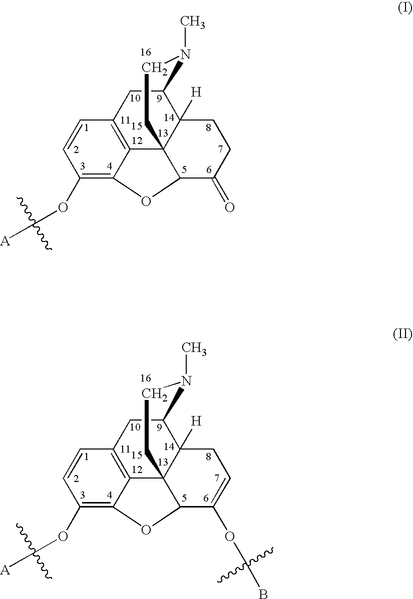
wherein,
A is selected from hydrogen, a carrier peptide, and a pharmaceutically acceptable salt of said carrier peptide;
B is selected from a carrier peptide, and a pharmaceutically acceptable salt thereof, with the proviso that A cannot be hydrogen in the compound of formula (I).

2. The compound of claim 1, wherein said carrier peptide is an amino acid.

3. The compound of claim 1, wherein said carrier peptide is selected from the group consisting of dipeptide, tripeptide, tetrapeptide and pentapeptide.

4. The compound of claim 1, wherein said carrier peptide is selected from the group consisting of Tyr-Tyr-Ile, Tyr-Tyr-Leu, Tyr-Tyr-Val, Gly-Gly-Ile, Gly-Gly-Leu, Gly-Gly-Val, Pro-Pro-Ile, Pro-Pro-Leu, Pro-Pro-Val, Lys-Lys-Ile, Lys-Lys-Leu, Lys-Lys-Val, Glu-Glu-Ile, Glu-Glu-Leu, Glu-Glu-Val, Phe-Phe-Ile, Phe-Phe-Leu, Phe-Phe-Val, Tyr-Tyr-Phe-Phe-Ile, Glu-Glu-Phe-Phe-Phe, Asp-Asp-Ile, and a salt thereof.

5. A composition comprising a compound of the formula I or the formula II: 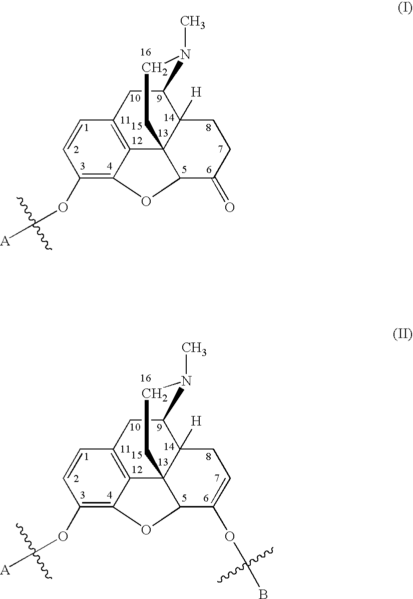
wherein,
A is selected from hydrogen, a carrier peptide, and a pharmaceutically acceptable salt of said carrier peptide;
B is selected from a carrier peptide, and a pharmaceutically acceptable salt thereof, with the proviso that A cannot be hydrogen in the compound of formula (I).

6. The composition of claim 5, wherein the compound or salt thereof provides a serum release curve that does not increase above the hydromorphone's toxicity level when taken at doses exceeding those within the therapeutic range for unbound hydromorphone.

7. The composition of claim 5, wherein the compound or salt thereof maintains a steady-state serum release curve that provides a therapeutically effective bioavailability but prevents spiking or increase blood serum concentrations compared to unbound hydromorphone.

8. The composition of claim 5, wherein when said composition is administered orally, bioavailability of the compound or salt thereof is maintained, and when administered intranasally, the bioavailability of said hydromorphone is decreased.

9. The composition of claim 5, wherein said composition is in a form suitable for oral administration.

10. The composition of claim 9, wherein said hydromorphone is resistant to release from said cater peptide when the composition is manipulated for parenteral administration.

11. A method of delivering hydromorphone comprising orally administering the composition of claim 5 to a patient.

12. A method of treating pain comprising orally administering the composition claim 5 to a patient.


SEQUENCE LISTING (27 sequences)

SEQ ID NO: 1 - protein, 4 aa, Artificial
  synthetic peptide
KYVI

SEQ ID NO: 2 - protein, 4 aa, Artificial
  synthetic peptide
YPVI

SEQ ID NO: 3 - protein, 5 aa, Artificial
  synthetic peptide
XEPPI

SEQ ID NO: 4 - protein, 5 aa, Artificial
  synthetic peptide
DDGGI

SEQ ID NO: 5 - protein, 5 aa, Artificial
  synthetic peptide
DDLLI

SEQ ID NO: 6 - protein, 5 aa, Artificial
  synthetic peptide
DDPPI

SEQ ID NO: 7 - protein, 5 aa, Artificial
  synthetic peptide
EEGGF

SEQ ID NO: 8 - protein, 5 aa, Artificial
  synthetic peptide
EELLL

SEQ ID NO: 9 - protein, 5 aa, Artificial
  synthetic peptide
EEFFL

SEQ ID NO: 10 - protein, 5 aa, Artificial
  synthetic peptide
EEFPI

SEQ ID NO: 11 - protein, 5 aa, Artificial
  synthetic peptide
EEPPL

SEQ ID NO: 12 - protein, 5 aa, Artificial
  synthetic peptide
EEPFI

SEQ ID NO: 13 - protein, 5 aa, Artificial
  synthetic peptide
EEEEI

SEQ ID NO: 14 - protein, 5 aa, Artificial
  synthetic peptide
EEFFF

SEQ ID NO: 15 - protein, 5 aa, Artificial
  synthetic peptide
GGEEI

SEQ ID NO: 16 - protein, 5 aa, Artificial
  synthetic peptide
KKLLI

SEQ ID NO: 17 - protein, 5 aa, Artificial
  synthetic peptide
KKPPI

SEQ ID NO: 18 - protein, 5 aa, Artificial
  synthetic peptide
FFEEI

SEQ ID NO: 19 - protein, 5 aa, Artificial
  synthetic peptide
FFFFF

SEQ ID NO: 20 - protein, 5 aa, Artificial
  synthetic peptide
TTGGI

SEQ ID NO: 21 - protein, 5 aa, Artificial
  synthetic peptide
TTFFI

SEQ ID NO: 22 - protein, 5 aa, Artificial
  synthetic peptide
YYLLI

SEQ ID NO: 23 - protein, 5 aa, Artificial
  synthetic peptide
YYFFI

SEQ ID NO: 24 - protein, 5 aa, Artificial
  synthetic peptide
YYPPI

SEQ ID NO: 25 - protein, 5 aa, Artificial
  synthetic peptide
YYPFI

SEQ ID NO: 26 - protein, 5 aa, Artificial
  synthetic peptide
YYFFI

SEQ ID NO: 27 - protein, 5 aa, Artificial
  synthetic peptide
YYFFV